(19) United States
(12) Patent Application Publication
(10) Pub. No.: US 2010/0028575 A1
(43) Pub. Date: Feb. 4, 2010

(54) HIGH INTEGRITY COMPOSITE BAGS ADAPTED FOR STEAM STERILIZATION

(76) Inventor: Steven Vanhamel, Velm (BE)

Correspondence Address: INTELLECTUAL PROPERTY / TECHNOLOGY LAW, PO BOX 14329, RESEARCH TRIANGLE PARK, NC, 27709, US

(21) Appl. No.: 12/441,717

(22) PCT Filed: Sep. 24, 2007
(86) PCT No.: PCT/US07/79316

§ 371 Date: Mar. 17, 2009

Related U.S. Application Data
(60) Provisional application No. 60/826,810, filed on Sep. 25, 2006.

(51) Int. Cl. B65D 30/08; A61L 2/07; B32B 27/32; B32B 37/00; B29D 22/00; B65D 33/16

(52) U.S. Cl. 428/35.2; 422/26; 156/281

(57) ABSTRACT

A sterilizable composite bag or pouch at a temperature of at least about 125° C. includes a first sheet comprising a gas-permeable but bacteria-impermeable polyolefin material, a second sheet comprising a first layer comprising a polyolefin with a density of less than 0.94 g/cm3 disposed between a second layer and a third layer comprising a high density polyolefin and having a density of at least 0.94 g/cm3, with the first sheet and the second sheet being locally thermally welded to one another to form a bag or pouch defining a cavity suitable for holding an article. A method includes thermally bonding such layers to form a plurality of bags and subjecting the plurality of bags to an autoclave steam sterilization process, wherein each second sheet thereafter remains pinhole-free and substantially impermeable to bacteria.

### STATEMENT OF RELATED APPLICATIONS

This application claims benefit of U.S. Patent Application No. 60/826,810 filed on Sep. 25, 2006.

### FIELD OF THE INVENTION

This invention generally relates to sterilizable bags and pouches into which an object may be introduced, sealed, and thereafter sterilized with a thermally-assisted sterilization process (e.g., utilizing steam at a temperature of about 125° C.), with the bag retaining high integrity despite being exposed to thermal stress.

### DESCRIPTION OF THE RELATED ART

Various products, including medical supplies and implantable medical materials, are desirably supplied in sterile form to a user. A desirable method for sterilizing products is to expose them to high temperature steam at a temperature of at least about 125° C. To ensure that sterility is not compromised between the time that the project is manufactured and the time it is packaged, sterilization is desirably performed on the product when it is disposed within a suitable package, and the package preferably is formed of materials that will maintain the product in sterile condition during and after shipment to the user.

The availability of membrane materials that are substantially impervious to bacteria and similar infectious agents, but that are permeable to sterilizing gases such as ethylene oxide and steam, has enabled the performance of gas-based sterilization on packaged products. Typically, such membrane material include a spun-bonded or melt-bonded polyolefin fiber web, for example, a spunbonded polyethylene marketed under the trade name capital Tyvek™, grade S-1073B from E.I. DuPont de Nemours, France. A typical steam sterilization product employed with packages fabricated at least in part with spunbonded or meltbonded material includes placing the packaged product in an autoclave, drawing vacuum to evacuate the package, and then introducing steam at a temperature of at least about 125° C. Such steam may enter and leave the pouch via the gas-permeable spunbonded or melt bonded sheet material, and thus sterilizing the interior of the bag and the product is those therein. Alternative sterilization means such as ethylene oxide and/or gamma radiation may be used, but steam sterilization is preferred in many applications.

It may not be feasible, however, to fabricate an entire package with spun bonded or melt bonded materials, for a variety of reasons. To begin with, such materials are difficult to join to one another with conventional means. Additionally, such materials are usually opaque in character, such that if a package is composed entirely of such materials, then the product packaged within cannot be visually identified or examined by the user without opening the package and potentially compromising the sterile character of the product. Furthermore, spun bonded or melt bonded materials are typically expensive, such that a package composed entirely of such materials might not be economical.

To overcome the limitations associated with fabricating a product package entirely of spun bonded or melt bonded materials, composite packages including such materials in part, and other materials in part, have been developed. For example, each of U.S. Pat. No. 6,279,745 to Huynen, et al. (“Huynen '745”), which is hereby incorporated by reference, describes a sterilizable composite package composed of a spunbonded gas-permeable polyolefin first sheet serving as a one face of a bag, and a second, multilayer gas-impervious polyolefin sheet serving as the other face of the bag. The first sheet may be composed of spunbonded polyethylene, such as Tyvek™, grade L1073B, and the second sheet may be composed of two, but more preferably three or more, layers of high density polyethylene (HDPE) or polypropylene. The two sheets may be heat sealed along three edges thereof to form and open pouch into which a product may be inserted, with the pouch being sealed along the fourth edge thereof to enclose the product in a gas-permeable sealed package amenable to gas sterilization. It is generally recognized that for a given material, density may be correlated to thermal resistance. Huynen '745 states that, for the non-permeable multilayer sheet, “[a] suitable density range is 0.94 to 0.96 g/cm³, preferably 0.955 g/cm³.” Huynen '745, column 3, lines 22-23. Huynen '745 goes on to teach that low-density polyethylene, or ultra low density polyethylene, sheet materials generally have two low a softening temperature to withstand steam sterilization at 125° C. or more. Huynen '745, column 3, lines 26-29. Similar disclosure is made in European Patent EP 0846445 (“Huynen '445”), issued to Messrs. Huynen (two of the co-inventors of Huynen '745), with such European Patent being incorporated by reference herein.

Applicant herein, who is a co-inventor with Messrs. Huynen of the subject matter claimed in Huynen '745, has substantial experience in fabricating composite packages according to the teachings of that patent. One limitation that has been discovered in steam sterilizing composite packages fabricated according to the teachings of Huynen '745—namely, including a three layer HDPE sheet peripherally sealed to a spun-bonded polyethylene (e.g., Tyvek™) sheet—is that the multi-layer HDPE sheet is relatively stiff or inflexible in character, and steam sterilization (i.e., with its attendant pressure/vacuum-induced and thermal-induced stresses) tends to cause the HDPE sheet to wrinkle and develop pinholes. These pinholes compromise the overall integrity of the bag and unacceptably elevate the risk that the interior of the bag and any contents disposed therein may be subject to microbiological contamination.

In light of these difficulties, it will be desirable to provide a polymeric bag formed from a gas-permeable spun-bonded or melt-bonded material joined to a substantially gas-impermeable sheet, with the resulting bag being adapted to withstand steam sterilization processes without elevating the risk of pinhole formation in the impermeable sheet.

### SUMMARY OF THE INVENTION

In one aspect of the invention, a composite bag or pouch comprises: (1) a first sheet comprising a polyolefin material adapted to permit the passage of a sterilization gas or vapor while being substantially impervious to bacteria; and (2) a second sheet comprising a plurality of bonded layers including a first layer comprising a polyolefin and having a density of less than 0.94 g/cm3 disposed between a second layer and a third layer comprising a high density polyolefin and having a density of at least 0.94 g/cm3; wherein the first sheet and the second sheet are locally thermally welded to one another to form a bag or pouch defining a cavity suitable for holding an article.

Another aspect of the invention relates to a method comprising the steps of: [A] thermally bonding a plurality of (1) first sheets comprising a polyolefin material adapted to permit the passage of a sterilization gas or vapor while being substantially impervious to bacteria, to (2) a plurality of substantially gas-impermeable second sheets comprising a plurality of bonded layers including a first layer comprising a polyolefin and having a density of less than 0.94 g/cm3 disposed between a second layer and a third layer comprising a high density polyolefin and having a density of at least 0.94 g/cm3, to form a plurality of individually and peripherally sealed composite bags each having an internal cavity suitable for holding an article, with the plurality of second sheets being substantially impermeable to bacteria; and [B] subjecting the plurality of bags to an autoclave steam sterilization process including exposing each bag of the plurality of bags to steam at a temperature of at least about 125° Celsius for a time sufficient to sterilize the internal cavity and any contents disposed therein; wherein, following the steam sterilization step, each second sheet remains pinhole-free and substantially impermeable to bacteria.

Other aspects, features and embodiments of the invention will be more fully apparent from the ensuing disclosure and any appended claims.

### BRIEF DESCRIPTION OF THE DRAWINGS

Embodiments of the invention may be best understood by referring to the following description and accompanying drawings, which illustrate such embodiments. In general within the drawings, like numbers are intended to refer to like elements or structures. Reference numbers are the same for those elements that are the same across different Figures. None of the drawings are drawn to scale unless indicated otherwise. In the drawings:

FIG. 1A illustrates a cross-sectional assembly view of a three layers—namely, a composite sheet formable from a central, flexible low density polyolefin core layer disposed between two high density polyolefin layers—suitable for forming a gas-impermeable composite layer useable in a high integrity composite bag adapted for steam sterilization.

FIG. 1B illustrates a cross-sectional view of a composite sheet assembled from the three layers of FIG. 1A.

FIG. 2A illustrates a front elevation view of a sterilizable bag according to one embodiment of the present invention, the bag having a gas impermeable composite sheet (such as the sheet of FIGS. 1A-1B) with localized thermally bonded seals along three peripheral sides thereof to a gas-permeable spun-bonded or melt-bonded sheet gas-permeable spun-bonded or melt-bonded sheet material.

FIG. 2B illustrates a cross-sectional view of the bag of FIG. 1A, with such cross-section taken along section lines “B”-“B.”

FIG. 2C illustrates the bag of FIGS. 1A-1B, further comprising a seal along a fourth peripheral side between the gas-impermeable composite sheet and the gas-permeable spun-bonded or melt-bonded sheet.

### DETAILED DESCRIPTION OF THE INVENTION, AND PREFERRED EMBODIMENTS THEREOF

The present invention is directed in various aspects to a composite bag or pouch suitable for sterilization, and related methods. A composite bag is formed from a gas-permeable (e.g., preferably nonwoven, such as spun-bonded or melt-bonded) polyolefin material sheet joined to a substantially gas-impermeable sheet comprising a first layer of a polyolefin having a density of less than 0.94 g/cm³ disposed between a second layer and a third layer comprising a high density polyolefin and having a density of at least 0.94 g/cm³. Polyethylene materials are preferred polyolefins for the various portions of the device, with spun-bonded or melt-bonded polyethylene film preferably used for the first sheet, high density polyethyelene (HDPE) film preferably used for the second and third layers of the second sheet, and low density polyethylene (LDPE) or metallocene linear low density polyethylene (LLDPE) films preferably used for the first layer (disposed intermediate to the second and third layers) of the second sheet. Although LDPE would appear to be unsuitable for steam sterilization because it has a melting temperature below the 125° C. target temperature of a steam sterilization cycle, the HDPE layers surrounding the LDPE core of the second sheet provide ample thermal resistance for the LDPE core to withstand such a cycle. The LDPE central layer is highly flexible, and permits the resulting three-layer composite sheet to resist stress-induced flexure during steam sterilization, and thus remain pinhole-free and substantially impermeable to bacteria.

References in the description to “one embodiment,” “an embodiment,” “a preferred embodiment,” and so on, indicate that the embodiment described may include a particular feature, structure, or characteristic, but every embodiment need not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments, whether or not explicitly described.

As used herein, the term “polymers” includes, but is not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the material. These configurations include, but are not limited to, isotactic, syndiotactic and atactic symmetries.

As used herein, the term “metallocene polymers” refers to those polymer materials that are produced by the polymerization of at least ethylene using metallocenes or constrained geometry catalysts, a class of organometallic complexes, as catalysts. As used herein, the terms “nonwoven” and “nonwoven web” refer to fibrous materials and webs of fibrous material, which are formed without the aid of a textile weaving or knitting process.

As used herein, the term “spunbonded fibers” refers to small diameter fibers, which are formed by extruding molten thermoplastic material as filaments from a plurality of fine capillaries of a spinnerette having a circular or other configuration, with the diameter of the extruded filaments then being rapidly reduced.

As used herein, the term “meltblown fiber” refers to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which may be to microfiber diameter (the average microfiber diameter is not greater than about 100 microns, for example, having an average diameter of from about 0.5 microns to about 50 microns, more particularly, microfibers may have an average diameter of from about 4 microns to about 40 microns).

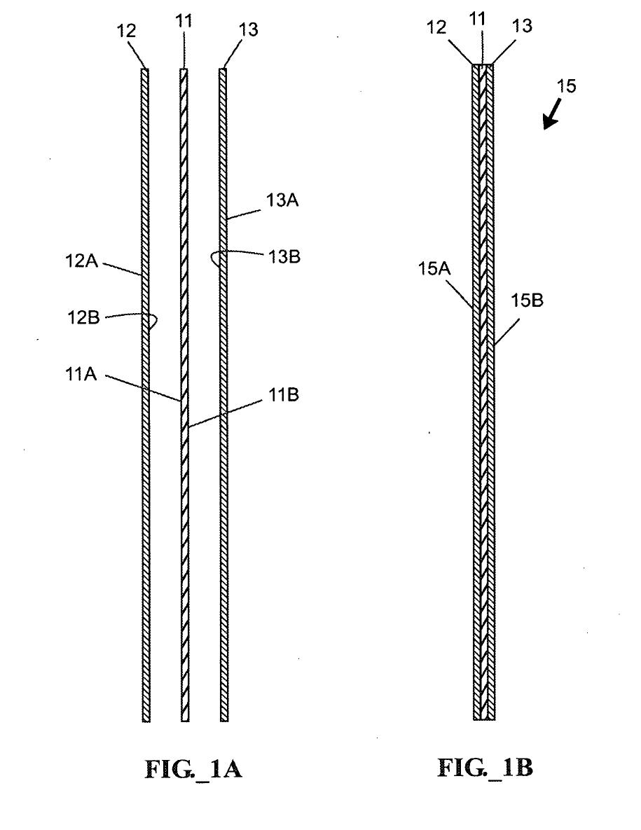

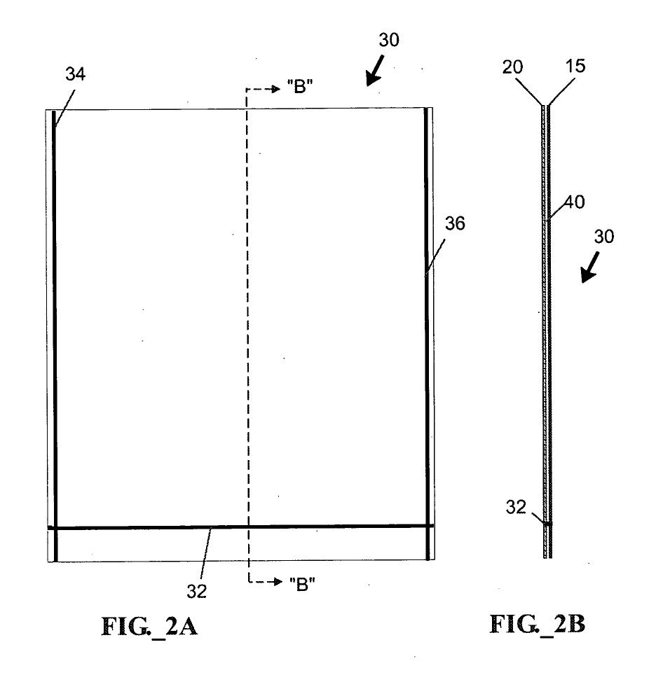

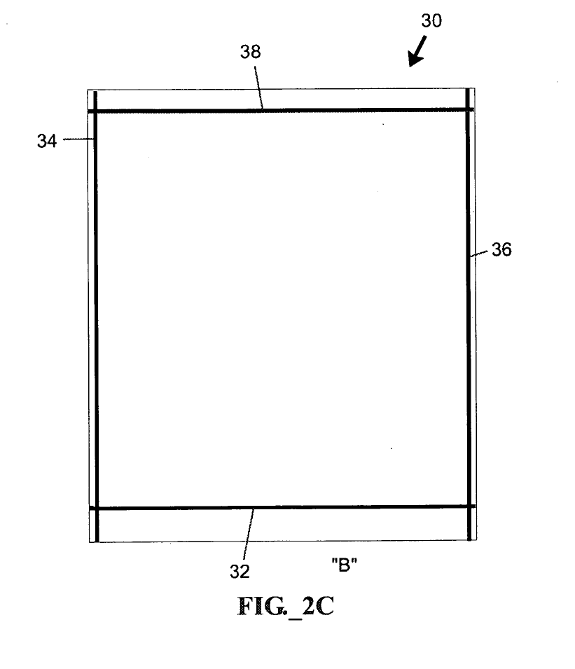

As indicated previously, one portion of a composite bag or pouch according to the invention includes a substantially gas-impermeable sheet comprising a first layer of a polyolefin having a density of less than 0.94 g/cm³ disposed between a second layer and a third layer comprising a high density polyolefin and having a density of at least 0.94 g/cm³. FIGS. 1A-1B illustrate such a sheet 15 composed of a first low-density core layer 11 disposed between a second layer 12 and a third layer 13 of a high-density polyolefin material. Each layer 11-13 has two faces, 11A-11B, 12A-12B, and 13A-13B, respectively. FIG. 1A shows the three layers 11-13 in assembly view. Such layers 11-13 are preferably thermally bonded along their faces, i.e., with one face 11A of the first layer 11 bonded to one face 12B of the second layer 12, and with the other face 11B of the first layer bonded to one face 13A of the third layer 13, to form a substantially contiguous composite sheet 15 having a first face 15A and a second face 15B, as shown in FIG. 1B. Any suitable heating method such as lamination, thermal heating between platens, or the like, may be employed to form the composite sheet 15, with such heating optionally being assisted with simultaneous application of pressure. Preferably, such heating is conducted at a temperature sufficient to melt at least a portion of the first core layer 11 to accomplish interpenetration of material along the faces 11A, 12B and 11B, 13A of the contacting layers 11, 12 and 11, 13, respectively. The resulting sheet 15 is suitable for localized joining, preferably by thermal means, to a gas-permeable first sheet to form a bag or pouch 30, such as illustrated in FIGS. 2A-2C. In one embodiment, the layers 11-13 comprise a forty micron thickness ULDPE first layer 11 bonded between thirty micron thickness second and third layers 12, 13 of HDPE. In another embodiment, the layers 11-13 include a fifty micron thickness ULDPE first layer 11 bonded between fifty micron thickness second and third layers 12, 13 of HDPE.

FIG. 2A illustrates a front elevation view of the sterilizable bag or pouch 30 composed of a gas-permeable sheet 20 and gas-impermeable composite sheet 15. As illustrated in FIG. 2A, the bag 30 includes local seals 32, 34, 36 adjacent to three peripheral sides thereof, leaving the two layers unsealed along a fourth side of the bag 30 to permit the insertion of an article to be sterilized into a cavity 40 (as illustrated in FIG. 2B) formed between the two sheets 15, 20. FIG. 2B illustrates the bag or pouch 30 of FIG. 2A in cross-sectional view taken along section lines “B”-“B,” and shows the seal 32 adjacent to a lower side of the bag or pouch. FIG. 2C illustrates the bag or pouch 30 of FIGS. 2A-2B in front elevation view, but with the bag further having a seal 38 adjacent to the fourth side thereof, to seal the cavity 40 (as illustrated in FIG. 2B) together with any article to be sterilized (not shown) disposed in the cavity 40. The resulting bag or pouch 30 permits the passage of gases or vapors (e.g., steam, ethylene oxide, and the like) into the cavity 40 through the porous sheet 20, but is substantially impermeable to bacteria and similar microbial contaminants. Such microbial impermeability persists even after the bag is autoclave steam sterilized at temperatures of at least 125° Celsius, as the composite layer 15 is highly resistant to pinhole formation under such conditions.

Applicant has conducted various tests to compare the integrity of steam sterilizable bags (“Conventional Bags”) each having an initially impermeable sheet composed entirely of HDPE versus steam sterilizable bags each having an impermeable composite sheet with a lower density core layer according to the present invention (“Inventive Bags”).

One comparison was performed with twenty samples each of three types of steam sterilizable bags composed of different starting materials but otherwise identical in configuration. The first bag type (“Conventional Bag 1”) included a permeable DuPont Tyvek™ grade S-1073B spunbonded polyethylene sheet having a thickness in a range of between 110-260 microns (per the manufacturer's specifications), locally and peripherally bonded with seals of at least 1.5 millimeters wide to a sheet composed of three sandwiched and face-bonded layers of twenty-seven micron thickness HDPE. The second bag type (“Inventive Bag 1”) included the same permeable DuPont Tyvek™ grade S-1073B spunbonded polyethylene sheet, but locally and peripherally bonded to a sheet composed of a forty micron thickness UDLPE layer sandwiched and face-bonded between two layers of thirty micron thickness HDPE. The third bag type (“Inventive Bag 2”) included the same permeable DuPont Tyvek™ grade S-1073B spunbonded polyethylene sheet, but locally and peripherally bonded to a sheet composed of a fifty micron thickness UDLPE layer sandwiched and face-bonded between two layers of fifty micron thickness HDPE. All sixty bags were subjected to autoclave steam sterilization including application of vacuum followed by introduction of steam at about 125° C. Following sterilization, the bags were tested for pinholes in the desirably gas-impermeable sheets. Of the twenty samples of the “Conventional Bag 1” type, ten were found to have developed pinholes in the sandwiched HDPE sheet. In contrast, no pinholes at all were found in the remaining forty samples including twenty bags of the type “Inventive Bag 1” and twenty bags of the type “Inventive Bag 2.”

Another comparison between bag types was performed with articles double packaged in steam sterilizable bags—i.e., with each article disposed in an inner steam sterilizable bag, and with each inner steam sterilizable bag disposed within an outer steam sterilizable bag. Ten double-bag samples each of two types of bags were prepared. One bag type (“Conventional Bag 2”) included a permeable DuPont Tyvek™ grade S-1073B spunbonded polyethylene sheet locally and peripherally bonded to a sheet composed of three sandwiched and face-bonded layers of HDPE. The other bag type (“Inventive Bag 3”) included the same permeable DuPont Tyvek™ grade S-1073B spunbonded polyethylene sheet material bonded to a gas-impermeable sheet composed of a ULDPE core layer sandwiched and face-bonded between two layers of HDPE. In each instance, an article to be sterilized was placed within a double bag of the applicable type—either “Conventional Bag 2” or “Inventive Bag 3”—with double-bagged samples prepared. Each article-containing double steam sterilizable bag was subjected to the same autoclave sterilization cycle of 122.0° C. for 60 minutes. After the steam sterilization cycle was completed, the article-containing bags were placed into shipping cartons and an ISTA 1A drop and shake test (including a vibration test according to the apparatus section of ASTM D 999-01 and a free fall drop text according to the apparatus section of ASTM D 5276-98. Thereafter, the articles were removed from the bags, and both pinhole and tensile pull tests were performed on the would-be impermeable layer portions of the bags. All bags tested passed the tensile pull test acceptance criteria of at least 20 N/15 mm. No pinholes were found on any of the formerly inner bags. Of the formerly outer bags, however, seven of the ten (i.e., 70%) purportedly gas-impermeable sheets from bags of the type Conventional Bag 2 exhibited pinholes, while none (i.e., 0%) of the gas-impermeable sheets of the type “Inventive Bag 3” exhibited any pinholes.

The resulting bag or pouch 30 is well-suited for steam sterilization, despite the use of low density polyolefin material within the multi-layer sheet 2, since the resulting sheet 2 has enhanced flexibility compared to a sheet composed of HDPE and thus resist formation of pinholes.

In view of the teachings of Huynen '745 and '755 that the suitable density range for the non-permeable multilayer sheet is between 0.94 to 0.96 g/cm³, and the fact that many low density polyolefin (e.g., low density polyethylene) materials have softening temperatures too low to withstand steam sterilization at about 125° C. or more, it is a surprising advantage of the present invention that a composite sheet incorporating low density polyethylene in a gas-impermeable sheet of a steam-sterilizable bag is not only workable, but also confers substantially improved performance in terms of eliminating pinholes that may be otherwise generated during steam sterilization.

While the invention has been described herein in reference to specific aspects, features and illustrative embodiments of the invention, it will be appreciated that the utility of the invention is not thus limited, but rather extends to and encompasses numerous other variations, modifications and alternative embodiments, as will suggest themselves to those of ordinary skill in the field of the present invention, based on the disclosure herein. Correspondingly, the invention as hereinafter claimed is intended to be broadly construed and interpreted, as including all such variations, modifications and alternative embodiments, within its spirit and scope.

1. A composite bag or pouch comprising:
a first sheet comprising a polyolefin material adapted to permit the passage of a sterilization gas or vapor while being substantially impervious to bacteria; and
a second sheet comprising a plurality of bonded layers including a first layer comprising a polyolefin and having a density of less than 0.94 g/cm³ disposed between a second layer and a third layer comprising a high density polyolefin and having a density of at least 0.94 g/cm³;
wherein the first sheet and the second sheet are locally thermally welded to one another to form a bag or pouch defining a cavity suitable for holding an article.

2. The bag or pouch of claim 1, wherein first sheet comprises a spun-bonded or melt-bonded polyolefin sheet material.

3. The bag or pouch of claim 1, wherein the first sheet comprises a spun-bonded or melt-bonded polyethylene sheet material.

4. The bag or pouch of claim 1, wherein the first sheet and the second sheet are peripherally sealed to define an internal cavity suitable for holding an article.

5. The bag or pouch of claim 1, wherein the first layer comprises a low density polyethylene.

6. The bag or pouch of claim 1, wherein the first layer comprises a metallocene linear low density polyethylene.

7. The bag or pouch of claim 1, wherein each of the second layer and the third layer comprises high density polyethylene having a density in a range of between about 0.94 g/cm³ and about 0.96 g/cm³.

8. The bag or pouch of claim 1, wherein the second layer has melting temperature below 125° Celsius.

9. A method comprising:
thermally bonding (i) a plurality of first sheets comprising a polyolefin material adapted to permit the passage of a sterilization gas or vapor while being substantially impervious to bacteria, to (ii) a plurality of substantially gas-impermeable second sheets comprising a plurality of bonded layers including a first layer comprising a polyolefin and having a density of less than 0.94 g/cm³ disposed between a second layer and a third layer comprising a high density polyolefin and having a density of at least 0.94 g/cm³, to form a plurality of individually and peripherally sealed composite bags each having an internal cavity suitable for holding an article, with the plurality of second sheets being substantially impermeable to bacteria; and
subjecting the plurality of bags to an autoclave steam sterilization process including exposing each bag of the plurality of bags to steam at a temperature of at least about 125° Celsius for a time sufficient to sterilize the internal cavity and any contents disposed therein;
wherein, following the steam sterilization step, each second sheet remains pinhole-free and substantially impermeable to bacteria.

10. The method of claim 9, wherein the plurality of bags comprises at least ten bags.

11. The method of claim 9, further comprising the step of thermally bonding the first layer, second layer, and third layer to form the plurality of second sheets.

12. The method of claim 9, wherein the plurality of first sheets comprise spun-bonded or melt-bonded polyolefin sheets.

13. The method of claim 12, wherein each sheet of the plurality of first sheets comprises polyethylene.

14. The method of claim 9, wherein the first layer comprises a low density polyethylene.

15. The method of claim 14, wherein the first layer comprises a metallocene linear low density polyethylene.

16. The method of claim 9, wherein each of the second layer and the third layer comprises high density polyethylene having a density in a range of between about 0.94 g/cm³ and about 0.96 g/cm³.

17. The method of claim 9, wherein the second layer has a melting temperature below 125° Celsius.

18. The method of claim 9, wherein the steam sterilization step includes applying subatmospheric pressure to the plurality of bags.
